(19) United States
(12) Patent Application Publication
(10) Pub. No.: US 2019/0353630 A1
(43) Pub. Date: Nov. 21, 2019

(54) SYSTEM FOR REMOTE GROUNDWATER MONITORING

(71) Applicant: Commonwealth Scientific and Industrial Research Organisation, Acton (AU)

(72) Inventors: Mikko VEPSÄLÄINEN, Elwood (AU); Kathryn LEVINGSTONE, Acton (AU); Justin SPANGARO, Hawthorn (AU)

(21) Appl. No.: 15/982,402

(22) Filed: May 17, 2018

(51) Int. Cl. G01N 33/18; E21B 47/06; E21B 47/12

(52) U.S. Cl. CPC G01N 33/1886; E21B 47/065; E21B 47/122; C02F 2209/02; G01V 9/02; C02F 2209/05; C02F 2209/04; C02F 2209/008; C02F 2209/06

(57) ABSTRACT

The invention is directed to a system for remote groundwater monitoring. The system includes one or more sensor modules configured for distribution in one or more groundwater monitoring wells. Each sensor module is adapted to acquire multi-parameter sensor data from each groundwater monitoring well. The multi-parameter sensor data includes electrochemical property data and electrical property data of groundwater in each groundwater monitoring well. One or more hubs are coupled to the one or more sensor modules for retrieving the multi-parameter sensor data and wirelessly communicating with an online server to upload the multi-parameter sensor data to the online server.

### RELATED APPLICATION

This application is related to Australian Patent Application No. 2018203504 filed on 17 May 2018, the entire contents of which are incorporated herein by reference.

### TECHNICAL FIELD

The invention is directed to a system for remote groundwater monitoring.

### BACKGROUND OF INVENTION

An aquifer is an underground layer of water-bearing permeable rock, rock fractures or unconsolidated materials (gravel, sand, or silt) from which groundwater can be extracted using a groundwater well.

Certain human activities, such as mining or farmland irrigation, as well as natural events such as rainfall can influence the properties of aquifers, such as depth or volume, salinity, acidity or the levels of other dissolved or suspended materials in the groundwater. Aquifers provide an important source of freshwater, and it is important to monitor and manage the state of health of aquifers, particularly where human activity is likely to impact the quality of groundwater.

It is often necessary to strictly regulate mining activities, which can cause significant impact to the environment, including potentially contaminating or degrading aquifers in or around the mine site. Most government agencies around the world will therefore require mining operators to adhere to approved monitoring and reporting regime so that any negative environmental impact can be either mitigated or identified and rectified. This monitoring and reporting regime will often apply to active mine sites, as well as closed mine sites until a period of time after the mining activities are complete, so as to ensure that the groundwater quality in and around the mine site remain unaffected and within approved parameters. In many cases, the monitoring and reporting regime may also be required before a new site can be exploited by mining activity.

In-Situ Recovery (ISR) mining involves drilling into an identified ore body (e.g. containing economically significant concentrations of metals such as copper or uranium) in the ground, and pumping solutions of metal-dissolving acids (“leachate”) into the ore body via an “injection well”. The outflow is pumped from nearby wells, and the resulting solution (“pregnant leach solution”) containing dissolved metals in solution form is extracted. The pregnant leach solution is ultimately processed to recover the dissolved metals.

Typically, the mining operations are designed to ensure that a negative pressure gradient exists from the surrounding aquifer to the mining area in order to prevent mining fluids from migrating outside the mining tenement via the aquifers. To ensure that the fluid movement remains appropriately controlled, regular monitoring of the aquifer water quality is performed via a series of groundwater monitoring wells surrounding the area being monitored. Ideally, the aquifer groundwater characteristics at the boundary of the tenement remain unaffected by the mining operations. Otherwise an “excursion event” has occurred and must be promptly rectified.

Conventionally, quality monitoring of aquifers in a remote mine site is typically performed by collecting groundwater samples from each groundwater monitoring well for analysis in a laboratory. In order to collect the groundwater samples, groundwater from each well must be pumped using specialised equipment installed in the well from a depth of typically 100 m, 200 m or more.

The specialist pumping equipment can be expensive to install and maintain, and are often prone to mechanical reliability and fouling problems. The manual labour required for routine groundwater sample collection and analysis can also be significantly time consuming and expensive, particularly when the mine site is remote and the distance between groundwater monitoring wells is considerable.

Moreover, manual groundwater sample collection from monitoring wells can be hazardous. Mines are often situated in remote and harsh locations that can be risky to access. In addition, the monitoring wells are often scattered in remote locations on the site. Accordingly, inherent workplace health and safety risks are associated with the manual activity of accessing each monitoring well and collecting the required groundwater samples.

In addition, manual groundwater sample collection from monitoring wells can also be unreliable. In particular, manual procedures are inherently prone to human error. Furthermore, adverse weather conditions can also make monitoring well sites inaccessible, potentially jeopardising mining operations if monitoring compliance requirements cannot be met. It can also be difficult to recruit trained and reliable sample collection technicians in many remote mining locations.

Sample processing and testing at a laboratory can also be time consuming and delays can undesirably impact the mine site's operations. For example, it can often take many weeks before samples are processed, potentially allowing an excursion even to go undetected for some time, thereby aggravating the problem by failing to facilitate an immediate response.

It is therefore desirable to provide a system for groundwater monitoring which overcomes or ameliorates one or more of the disadvantages or problems described above, or which at least provides the consumer with a useful choice.

### SUMMARY OF INVENTION

According to one aspect of the invention, there is provided a system for remote groundwater monitoring, the system including

one or more sensor modules configured for distribution in one or more groundwater monitoring wells, each sensor module being adapted to acquire multi-parameter sensor data from each groundwater monitoring well, wherein the multi-parameter sensor data includes electrochemical property data and electrical property data of groundwater in each groundwater monitoring well,

one or more hubs coupled to the one or more sensor modules for retrieving the multi-parameter sensor data and wirelessly communicating with an online server to upload the multi-parameter sensor data to the online server.

The one or more groundwater monitoring wells may be located in or around a mine site, and the one or more sensor modules may be configured to be submerged underwater to acquire multi-parameter sensor data from an aquifer layer of the groundwater monitoring wells. In one embodiment, the mine site is an in-situ recovery mine site.

Advantageously, the sensor modules is capable of automatically retrieving any suitable multi-parameter sensor data as required over any suitable monitoring period without the need for manual sampling after the system is installed. The collection of live sensor data across all different groundwater monitoring wells concurrently provides high fidelity measurement of the groundwater properties to properly monitor the health of groundwater on site, and allow prompt detection of any problems so that an appropriate response can be timely formulated.

Typically, each sensor module includes solid-state sensors comprising a reference electrode, a counter electrode and one or more working electrodes. In particular, each sensor module may include a plurality of solid-state sensors comprising a pH electrode, and a reference electrode, for acquiring data relating to electrochemical properties of the groundwater in each groundwater monitoring well. In addition, each sensor module may further include an oxidation reduction potential electrode. Moreover, each sensor module may further include an electrical conductivity sensor for acquiring data relating to electrical properties of the groundwater in each groundwater monitoring well. The sensor module may further include a temperature sensor.

Each sensor module may allow coupling of a water pressure sensor. In particular, each sensor module may be configured to allow connection of one or more self-contained sensor units. The one or more sensor units may include a water pressure sensor unit.

The system may further include a barometer located within 20 km of the one or more groundwater monitoring wells for measuring atmospheric pressure, the barometer being configured for wireless communication with the online server, the system being configured to determine the water level in the one or more groundwater monitoring wells based on the measurements from the barometer and the one or more water pressure sensors.

Alternatively, the system may further include a reference water pressure sensor for distribution in the one or more groundwater monitoring wells such that the reference water pressure sensor is suspended above groundwater, the reference water pressure sensor being coupled to the hub for wireless communication with the online server, the system being configured to determine the water level in the one or more groundwater monitoring wells based on the measurements from the reference water pressure sensor and the one or more water pressure sensors.

In in-situ recovery mine sites, it has been determined that changes in groundwater pH and electrical conductivity can often provide a reliable indication of a potential leach problem or an “excursion event”. The temperature sensor may be used to provide measurements of temperature to indicate reliability of pH measurements. Moreover, pressure, pH and electrical conductivity measurements can be analysed separately or in combination with each other to detect any anomalous conditions which can provide an indication of an “excursion event”.

In one embodiment, each sensor module includes a pH sensor for measuring electrochemical property data of groundwater, and an electrical conductivity sensor for measuring electrical property data of groundwater in each groundwater monitoring well. In one embodiment, each sensor module may include an oxidation reduction potential sensor for measuring electrochemical property data of groundwater, and an electrical conductivity sensor for measuring electrical property data of groundwater in each groundwater monitoring well.

The one or more hubs may be adapted to periodically sample sensor data from each sensor module for wireless communication to the online server in real-time or near real-time. The provision of real-time or near real-time sensor data further allows rapid detection of changes of groundwater properties across the different groundwater monitoring wells, so as to allow an “excursion event” to be immediately identified for rectification.

Each sensor module may be configured to measure electrical conductivity of groundwater and electrochemical properties of groundwater in each respective groundwater monitoring well at different and non-overlapping time intervals. Arranging the measurement of electrochemical properties and electrical conductivity properties in this manner effectively allows the sensor module to avoid a current used for the electrical conductivity measurement from interfering the electrochemical measurements in groundwater.

Each sensor module may be associated with a spacer for spacing a sensor face of the sensor module at a predetermined distance away from a wall of a respective groundwater well. The spacer may separate the sensor face from the wall of the respective groundwater well by any suitable distance to reduce interference. In one embodiment, the spacer spaces the sensor module at a distance of roughly 14 mm or more away from a side wall of a respective groundwater monitoring well. In other embodiments, the distance may be roughly 12 mm or more, or 10 mm or more, or 8 mm or more, or 6 mm or more, or 4 mm or more, or 2 mm or more.

The system may be configured to calibrate a measurement of electrical conductivity acquired by a sensor module based on a predetermined interference value. The calibration may include subtracting a predetermined interference value from a measured electrical conductivity value.

The one or more hubs may be configured to provide power to the one or more sensor modules, schedule sampling of the one or more sensor modules, buffer sensor data from the one or more sensor modules, and upload the sensor data to the online server.

The online server may be configured to receive sensor data from the hub, store the received sensor data in a database, and provides online access of the sensor data to a remote user.

The system may include any suitable number of hubs. In one embodiment, for example, if two or more monitoring wells are located in close proximity to one another, the one or more sensor modules in deployed in each of the closely located monitoring wells may be coupled to a shared hub. The system may include any number of shared hubs depending on site requirements and monitoring well locations. In some embodiments, for example, if some monitoring wells are located far away from one another, the one or more sensor modules deployed in each monitoring well may be coupled to a respective hub for the monitoring well. The system may include any number of individual hubs and/or shared hubs in combination depending on site requirements and monitoring well locations.

The system may further include an interface for graphically presenting the multi-parameter sensor data. For example, the interface may present heat maps, graphs and the like, or any combination thereof to provide useful interpretation of the sensor data. The interface may also provide an alert if a rapid change in any one or more of the multi-parameter sensor data measurement is detected.

According to another aspect of the invention, there is provided a groundwater monitoring system including

one or more sensor modules configured for distribution in one or more groundwater monitoring wells, the groundwater monitoring wells being located in or around an in-situ recovery mine site,

the one or more sensor modules being configured to be submerged underwater to measure multi-parameter sensor data from an aquifer layer of the groundwater monitoring wells, wherein the multi-parameter sensor data includes electrochemical property data and electrical property data of groundwater in each groundwater monitoring well,

one or more hubs coupled to the one or more sensor modules, the hubs being configured to periodically sample the multi-parameter sensor data measured by the one or more sensor modules, and

an online server for receiving and storing the sensor data uploaded from the hub and providing online access to the sensor data.

Each sensor module may be configured to measure electrical conductivity of groundwater and electrochemical properties of groundwater in each respective groundwater monitoring well at non-overlapping time intervals. The electrochemical properties may include pH values.

Each sensor module may include one or more sensors for measuring electrochemical properties of groundwater in each groundwater monitoring well, and an electrical conductivity sensor for measuring conductivity of the groundwater in each groundwater monitoring well, wherein the sensors for measuring electrochemical properties and the electrical conductivity sensor are embedded in the sensor module. The sensors for measuring electrochemical properties may include an oxidation reduction potential electrode, a pH electrode, and a reference electrode, for acquiring data relating to electrochemical properties of the groundwater in each groundwater monitoring well. Each sensor module may further include a temperature sensor.

According to yet another aspect of the invention, there is provided a sensor module for a system as described in the preceding paragraphs.

According to a further aspect of the invention, there is provided a sensor module for groundwater monitoring, the sensor module being configured to be submerged underwater to measure multi-parameter sensor data from an aquifer layer of groundwater monitoring wells in or around an in-situ recovery mine site, wherein the sensor module includes one or more sensors for measuring electrochemical properties of groundwater in each groundwater monitoring well, and an electrical conductivity sensor for measuring electrical conductivity of the groundwater in each groundwater monitoring well, wherein the sensors for measuring electrochemical properties and the electrical conductivity sensor are embedded in the sensor module.

The sensor module may be configured to measure electrical conductivity of groundwater and electrochemical properties of groundwater in each respective groundwater monitoring well at non-overlapping time intervals.

The sensors for measuring electrochemical properties may include an oxidation reduction potential electrode, a pH electrode, and a reference electrode, for acquiring data relating to electrochemical properties of the groundwater in each groundwater monitoring well. The sensor module may further include an embedded temperature sensor.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristic described herein may be combined in any suitable manner in one or more combinations.

In order that the invention may be more readily understood and put into practice, one or more preferred embodiments thereof will now be described, by way of example only, with reference to the accompanying drawings.

### BRIEF DESCRIPTION OF DRAWINGS

FIG. 1 is a schematic diagram of a groundwater monitoring system according one embodiment of the present specification.

FIG. 2 is a schematic diagram of the network architecture of the groundwater monitoring system of FIG. 1.

FIG. 3 shows a sensor module of the ground water monitoring system illustrated in FIG. 1.

FIG. 4 is a schematic block diagram of the circuit layout for the sensor module as shown in FIG. 3.

FIG. 5 is an end view of a sensor module according to an embodiment of the invention deployed in a groundwater monitoring well.

FIG. 6 is a line graph showing the relationship between electrical interference and the distance between a face of the sensor module and a conductive material.

FIG. 7 illustrates a sensor module assembly including the sensor module shown in FIG. 3 and a spacer in the form of a sleeve or cone fitted around the body of the sensor module.

### DETAILED DESCRIPTION

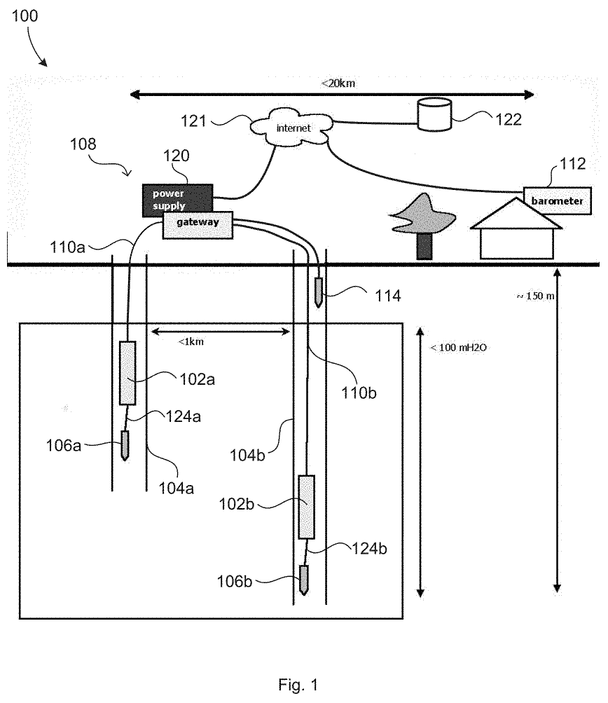

FIG. 1 illustrates a groundwater monitoring system 100 including sensor modules 102a, 102b. Each sensor module 102a, 102b is located in a respective groundwater monitoring well 104a, 104b. In one embodiment, the groundwater monitoring wells are located in or around an in-situ recovery mine site. Two sensor modules 102a, 102b are shown in FIG. 1 for illustrative purposes only. Depending on the application, the monitoring system 100 can have any suitable number of sensor modules 102 based on the total number of groundwater monitoring wells at a particular site. Typically, one sensor module 102 will be deployed in each respective groundwater monitoring well.

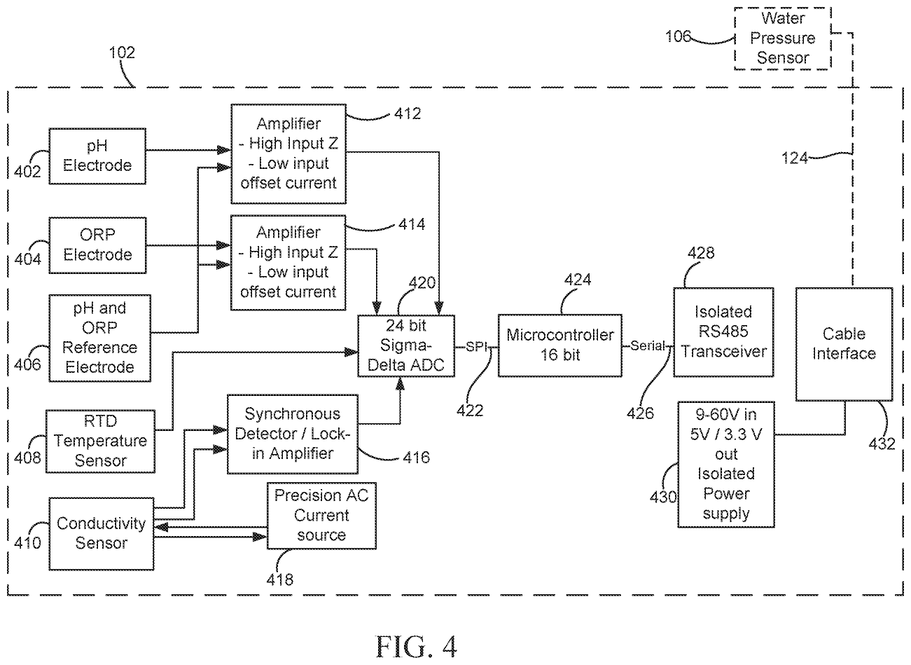

Each sensor module 102a, 102b includes a plurality of solid state sensors and is configured to be submerged underwater to measure multi-parameter sensor data from an aquifer layer of the groundwater monitoring wells 104a, 104b. Typically, each sensor module 102a, 102b includes a plurality of sensors for measuring properties of groundwater in each groundwater monitoring well 104a, 104b. As will be discussed in more detail below with reference to FIG. 4, each sensor module 102 includes a reference electrode 406, oxidation reduction potential (ORP) electrode 404, a pH electrode 402, electrical conductivity sensor 410, and a temperature sensor 408. In some embodiments, the sensor module 102 may further include a metal ion sensor (not shown) for detecting certain metal ions in the groundwater.

The system 100 further includes a hub 108 connected to the sensor modules 102a, 102b via cables 110a, 110b. The hub 108 is configured for wireless communication with an online server 122, typically via internet connection 121.

Each sensor module 102a, 102b is also configured to allow expansion to interface with other like sensor modules or self-contained sensor units 106a, 106b in alignment with the cabling for the respective sensor module 102a, 102b. The in-line arrangement of the sensor modules 102a, 102b and respective sensor units 106a, 106b advantageously provides a streamlined sensor assembly profile which minimises the likelihood of any part of the sensor assembly from being jammed during deployment or retrieval in a groundwater monitoring well.

In this embodiment, the self-contained sensor unit 106 is a water pressure sensor connected to a lower end of a respective sensor module 102. The water pressure sensor 106a, 106b may be an off-the-shelf water pressure sensor connectable to the respective sensor module 102a, 102b via a respective RS-485/MODBUS connection 124a, 124b.

Measurements from the water pressure sensors 106a, 106b can be calibrated based on measurements for atmospheric pressure to provide measurements for water level within each well 104a, 104b.

To provide the reference atmospheric pressure data, the system 100 provides a barometer 112 configured for wireless communication with the online server 122. The barometer 112 is preferably located within a 20 km radius of the wells 104a, 104b to provide accurate atmospheric pressure relative to the well locations 104a, 104b. Alternatively, the system 100 can provide a water pressure sensor 114 for locating above groundwater in any one of the groundwater monitoring wells 104b to provide a reading for atmospheric pressure.

The hub 108 is configured to conduct scheduled periodic sampling of the sensor modules 102a, 102b, sensor units 106a, 106b, and reference water pressure sensor 114, buffer sensor data, and upload the sensor data to the online server 122. The online server 122 receives and stores the sensor data uploaded from the hub 108 and provides online access to the sensor data.

The hub 108 is also configured to provide power to the sensor modules 102a, 102b, sensor units 106a, 106b, and reference water pressure sensor 114 via power supply 120. In the embodiment shown, the power supply 120 typically includes a solar power unit and a battery (not shown) as the groundwater monitoring wells for an in-situ recovery mine site are generally remote and without access to a power grid. However, it will be appreciated that any suitable renewable and/or fossil fuel power source may be used, for example the power source may include mains power supply, one or more power generators, batteries, solar power units, wind power, hydro power, wave power, geothermal power and the like, or any combination thereof.

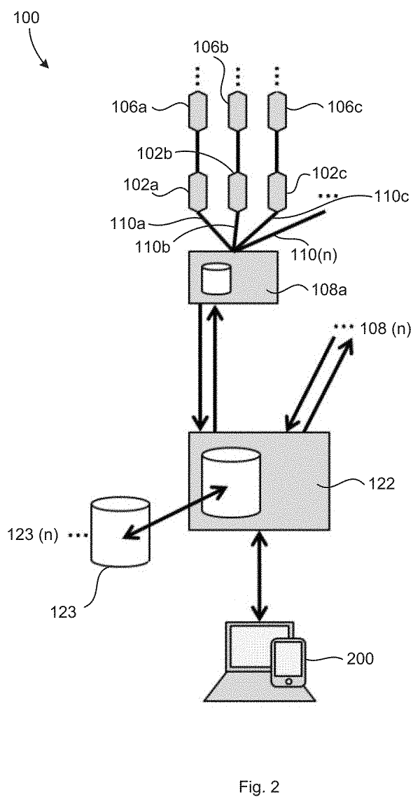

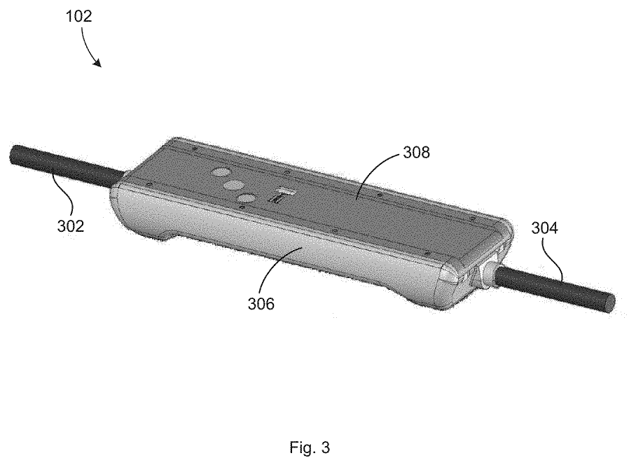

FIG. 2 is a schematic diagram which illustrates the network architecture for the system 100 for groundwater monitoring according to an embodiment of the present invention. The system 100 includes a plurality of sensor modules 102a, 102b, 102c-102(n) for measuring groundwater properties for each groundwater monitoring well 104. Further detail of the sensor modules 102 will be described below with reference to FIGS. 3 to 4. Whilst only three sensor modules 102a, 102b, 102c are shown in FIG. 2, it will be understood that the system 100 can be expanded to include any suitable number of sensor modules 102, to coincide with the number of monitoring wells 104 at a particular site.

Each sensor module 102a, 102b, 102c-102(n) can be expandable for interface with a self-contained sensor unit 106a, 106b, 106c-106(n) such as a self-contained water pressure sensor as shown in FIG. 1, or any other type of sensor unit depending on the nature of the application (e.g. the type of mining activity involved and the groundwater properties which require monitoring). In some embodiments, each sensor module 102a, 102b, 102c-102(n) may be expanded to interface with any number of like sensor modules 102a, 102b, 102c-102(n) so as to provide sensor strings of any suitable length.

Preferably, each of the sensor modules 102a, 102b, 102c-102(n) include non-volatile storage for its unique metadata. Prior to deployment, an operator may calibrate a sensor module 102a, 102b, 102c-102(n) against reference parameters and program metadata (including transducer drive parameters, ADC gains, calibration coefficients and locational coordinates) into the sensors 102a, 102b, 102c-102(n).

The metadata that may be stored on the electronics module within the sensor modules 102a, 102b, 102c-102(n) may include: sensor module model/variant ID, unique serial number, transducer drive parameters (e.g. excitation voltages or currents, ADC gains, oversampling factors), coefficients for conversion of raw ADC values or potentials into calibrated physical units, calibration date/time (UTC), locational coordinates, and a cyclic redundancy check (CRC) or hash of the foregoing metadata for verification purposes.

The electronics module (see FIG. 4) controls power output to the sensors, signal conditioning and digitisation, correction & unit conversion, and communicates data to the hubs 108a-108(n). Preferably, the basic data processing will be carried out in the sensors, for example: oversample averaging and calculating the standard deviation of the signal over the sampling time, current and voltage data of the conductivity sensor is processed into the resistance values, resistance values of the temperature sensors, millivolt readings of the potentiometric sensors, simultaneous correction & conversion to real-world units, from stored calibration coefficients.

The sensors 102a, 102b, 102c-102(n); 106a, 106b, 106c-106(n) are wired together via use of data cables 110a, 110b, 110c-110(n), allowing power transfer and digital data transfer between the hub 108a-108(n) and the sensors 102a, 102b, 102c-102(n); 106a, 106b, 106c-106(n). In the embodiment shown, each sensor module 102a, 102b, 102c-102(n) and the respective sensor unit 106a, 106b, 106c-106(n) is connected to a hub 108 via cabling 110a, 110b, 110c-110(n). The hub 108 communicates via a wired or wireless network interface with the server 122 which includes a database 123 for storing data received from the sensors 102a-102(n), 106a-106(n) along data cable 110a-110(n).

In some embodiments, particularly if the location of the site and distance between groundwater monitoring wells 104 do not practically permit wired connection between the sensor modules 102a, 102b, 102c-102(n), each sensor module 102a, 102b, 102c-102(n) and the respective sensor unit 106a, 106b, 106c- 106(n) can be connected to a respective hub 108a-108(n). Each hub 108a-108(n) communicates via a wired or wireless network interface with the server 122.

An end user may interact with the sensor data stored in the database 123 via the user interface of a PC and/or mobile device 200. Typically, the PC and/or mobile device 200 accesses the online server 122 via any suitable network. The device 200 may take any suitable form such as a computer, mobile communication device, tablet or the like. In some embodiments, the hub 108 or hubs 108a-108(n) may be a gateway or a data logger.

In this embodiment, the wireless network communication between the hub 108 and the online server 122, the online server 122 and the device(s) 200 is the internet 121. In some embodiments, the network may include a local area network. The device 200 may access the network in any suitable manner such as via Wi-Fi, 3G, 4G or satellite and the like, using any suitable protocol and data format. Each hub 108a-108(n) may initiate a connection to the server 122 and ‘push’ data, or the server 122 may initiate a connection to each hub 108a-108(n) and ‘pull’ data. Preferably, each hub 108a-108(n) is adapted to periodically sample sensor data from each of the respective sensor modules 102a, 102b, 102c-102(n) and respective sensor units 106a, 106b, 106c-106(n) for communication to the online server in real-time or near real-time.

The server 122 receives data from the hubs 108a-108(n) and stores the data for retrieval in a database 123. The data may be further processed on the server 122 or in a database 123a to 123(n) which is in a geographically redundant location. Additionally, geographic redundancy of the servers may be provided which allows a gateway to fail-over to a secondary or tertiary server if one is unreachable. A user associated with device 200 may, if authorised, and depending on their role, access the data on the server 122 and database 123 and may acquire visualisation and analysis of the data in a user friendly format. Typically, the sensor data may be presented to the user graphically. For example, in the form of graphs, heat maps and the like. In one embodiment, the system 100 is also configured to detect rapid change in the sensor data collected by the sensor modules 102a-102(n) and alert a user via the user interface 200.

In some embodiments, the hubs 108a-108(n) include the capability to re-write metadata and re-program the embedded firmware on attached sensors, when instructed to do so by an authorised administrator user through the user interface via a device 200 or the like.

Now referring to FIG. 3, which illustrates the external appearance of a sensor module 102. The sensor module 102 is configured for expansion to interface with additional like sensor modules 102 or self-contained sensor units, such as a water pressure sensor 106 as shown in FIG. 1. In particular, connection interface 302, 204 allow additional sensor modules 102 or other sensor units 106 to be coupled to either or both ends of the sensor module 102 in a streamlined arrangement, so as to facilitate deployment in a well. In some embodiments, the sensor module 102 may be expandable to interface with a metal ion sensor.

The sensor module 102 includes a robust protective and waterproof casing 306 to protect the sensor electronics during operation, particularly under high water pressure environments. The casing 306 is preferably made from a suitable acid resistant plastic, such as PMMA or suitable epoxy, and the solid-state sensors are preferably mounted into the casing using an acid resistant polymer.

The sensor module 102 includes a sensor face 308 through which the sensors take measurements from the surrounding groundwater.

Now referring to FIG. 4, which is a schematic block diagram illustrating the circuit layout of sensor module 102. The sensor module 102 includes a pH electrode 402, an oxidation reduction potential (ORP) electrode 404 and a reference electrode 406 and potentiostats. The three electrodes 402, 404, 406 conduct measurements of electrochemical properties of the groundwater. Measurements from the pH electrode 402 is taken against the potential of the reference electrode 406 and amplified via amplification module 412. Similarly, measurements from the ORP electrode is taken against the potential of the reference electrode 406 and amplified via amplification module 414. The electrodes 402, 404, 406 therefore measure the pH and ORP of the groundwater. The pH electrode 402 measures the acidity and alkalinity of the water. The ORP electrode measures the amount of contaminant in the water by measuring the dissolved oxygen. A lower ORP level indicates more contaminants in the water as organics are consuming the oxygen resulting in less dissolved oxygen, and vice versa.

The sensor module 102 further includes temperature sensor 408, for example, in the form of a resistance temperature detector (RTD) and a conductivity sensor 410. The AC current source 418 provides an AC signal to excite a probe of the conductivity sensor 410 for measurement of electrical conductivity of the groundwater. The electrical conductivity (EC) output signal from the conductivity sensor 410 is therefore in the form of a modulated signal. A Synchronous detector or Lock-in amplifier module 416 is used to extract the EC measurement information from the modulated signal.

Processed sensor data from sensor devices 402, 404, 406, 408, 410 is digitised using an Analogue to Digital Converter (ADC) 420. The digital sensor data is received by a microcontroller 424 via a Serial Peripheral Interface bus (SPI) 422. The microcontroller 424 outputs the digital sensor data via a serial connection with a transceiver 428 for transmitting the sensor data via cable interface 432 to a respective hub 108 (see FIGS. 1 and 2).

The sensor module 102 further includes a power supply circuit 430 for interfacing with the power supply 120 (see FIG. 1) via the cable interface. The power supply circuit 430 conditions the incoming power signal from the power supply 120 for application to electronics of the sensor module 102.

In a preferred embodiment, the cable interface 432 provides the RS485/MODBUS interface with external sensor units, such as the water pressure sensor 106. One or more additional like sensor modules 102 and/or external self-contained sensor units can therefore be connected to the hub 108 via cable interface 432.

It has been identified that simultaneous measurement of electrical conductivity (EC) and electrochemical properties (such as pH and ORP) can be problematic. In particular, as a current is passed through the solution to conduct the EC measurement, the current can interfere with the electrochemical measurements using the pH 402, ORP 404 and reference 406 electrodes.

The microcontroller 424 therefore schedules operation of the electrodes 402, 404, 406 and the conductivity sensor 410 such that the sensor measurements via electrodes 402, 404, 406 are taken at different time to the sensor measurements from the conductivity sensor 410. By scheduling sensor operation so that sensor measurements are taken at different points in time, in a time-share arrangement, interference between the sensor measurements can be effectively mitigated.

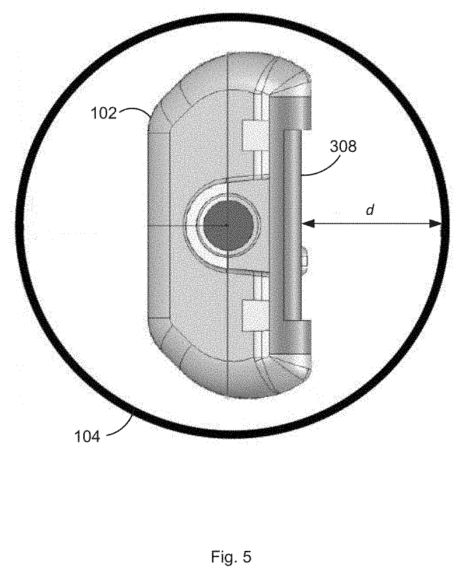

It has been further identified that the EC measurement can be subject to further interference, for example, if a conductive or insulating material is present and located in proximity to the sensor face 308. Accordingly, when the sensor module 102 is deployed in a groundwater monitoring well 104, the sidewalls of the well 104 may interfere with the EC sensor 410 measurements. As shown in FIG. 5, a distance between the sensor face 308 of sensor module 102 deployed in a well 104 is represented by ‘d’. It has been determined that a distance of roughly 14 mm or more between the sensor face and a sidewall of the groundwater monitoring well 104 will be sufficient to render any interference in the EC sensor 410 reading due to side walls of the well 104 negligible.

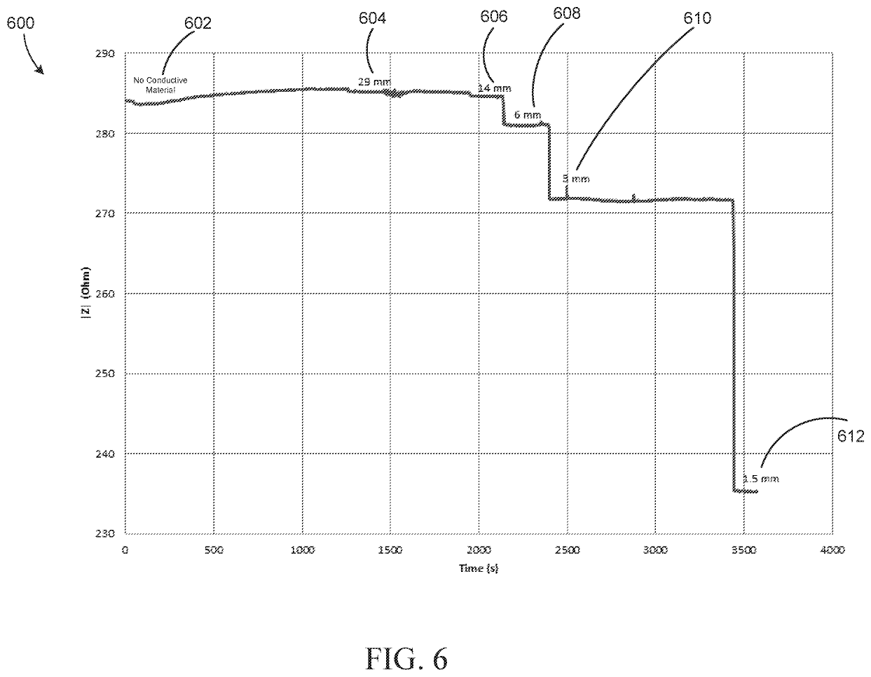

FIG. 6 in a line graph 600 illustrating the effects of EC measurements in a fluid in the presence of a conductive material. The y-axis of the graph 600 provides a measurement of impedance (Z) in ohms and the x-axis of the graph 600 provides a measurement of time (T) in seconds.

In a laboratory environment, EC measurements of water were taken using the sensor module 102 in the presence of a conductive material (e.g. a piece of conductive metal). The EC measurements were taken over time (roughly 3600 seconds) whilst moving the conductive material closer to the sensor face 308 of the sensor module 102. In particular:
Portion 606 of the EC measurement represents the conductivity of water with no conductive material present. The measured impedance is roughly 285 omhs.
Portion 604 of the EC measurement is conducted in the presence of a conductive material at a distance of 29 mm from the sensor face 308. The impedance remains roughly unchanged at approximately 285 omhs.
Portion 604 of the EC measurement is conducted in the presence of the conductive material at a distance of 14 mm from the sensor face 308. The impedance remains roughly unchanged at approximately 285 omhs.
Portion 604 of the EC measurement is conducted in the presence of the conductive material at a distance of 6 mm from the sensor face 308. The impedance reduces to roughly 281 omhs resulting in an increase in electrical conductivity.
Portion 604 of the EC measurement is conducted in the presence of the conductive material at a distance of 3 mm from the sensor face 308. The impedance is further reduced to roughly 272 omhs resulting in a further increase in electrical conductivity.
Portion 604 of the EC measurement is conducted in the presence of the conductive material at a distance of 1.5 mm from the sensor face 308. The impedance further reduces to roughly 235 omhs resulting in a further increase in electrical conductivity.

Graph 600 therefore illustrates that when the conductive material is positioned at 14 mm or more away from the sensor face 308, negligible interference from the conductive material is recorded during EC measurements of the sensor module 102.

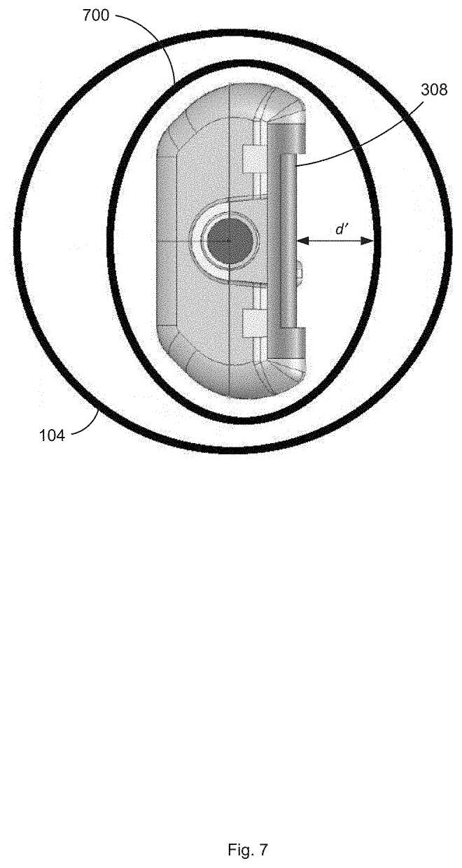

Each sensor module 104 can therefore be fitted with a spacer (not shown) for spacing the sensor face 308 away from side walls of the wells 104 during use. Any suitable spacing mechanism can be used. In one embodiment as shown in FIG. 7, a sleeve or cone 700 can be fitted around the body of the sensor module 102 to provide the minimum separation (d′) of 14 mm between the sensor face 308 and a side wall of the well 104.

In an alternative embodiment, the typical interference caused by side walls of groundwater monitoring wells having a particular diameter at a particular site can be predetermined in a simulation or laboratory environment. Each sensor module 102 can be pre-calibrated based on the predetermined interference values for each monitoring well before deployment to account for the likely interference values. For example, by subtracting the predetermined interference values from the measured EC values.

The system 100 and sensor modules 102 of the present invention therefore provides significant advantages over traditional manual sampling methods of groundwater monitoring. The instantaneous, automatic and real-time nature of the system 100 and associated sensor modules 102 is capable of detecting any notable changes in one or more parameters at more than one well locations simultaneously. When the captured sensor data is correlated, the system 100 is capable of providing a strong indication of an excursion event in a timely manner. The relative values, and rates of change of the affected parameters may provide further insight into the characteristics of the excursion event, such as direction, extent or rate. Such insights are often difficult to obtain via monitoring data that is sparse and where samples across different monitoring wells are not taken at the same time.

In addition, the sensor module 102 is capable of instantaneously conducting measurements of electrochemical properties of the water concurrently with measurements of temperature and electrical conductivity without interference. The sensor module 102 includes a plurality of embedded sensor electronics to simplify installation procedures, and allow the provision of streamlined and compact sensor module assemblies for deployment in groundwater monitoring wells at great depths of typically 100 to 300 m or more. Providing the different sensor electronics on board also allows the microcontroller 424 to provide appropriate scheduling and control of the sensor electronics to provide high fidelity and reliable sensor measurements.

The network architecture of the system 100 (see FIGS. 1 and 2) also advantageously enables the captured sensor data to be transmitted to a technician via interface 200 in real time or near real time so that any rapid changes in the monitored water properties can be captured and identified immediately without delay.

The foregoing embodiments are illustrative only of the principles of the invention, and various modifications and changes will readily occur to those skilled in the art. The invention is capable of being practiced and carried out in various ways and in other embodiments. It is also to be understood that the terminology employed herein is for the purpose of description and should not be regarded as limiting.

In the specification, including the claims, the term “comprise”, and variants of that term such as “comprises” or “comprising”, are used to mean “including but not limited to”, unless expressly specified otherwise, or unless in the context or usage an exclusive interpretation of the term is required.

Reference to any background art or prior art in this specification is not an admission such background art or prior art constitutes common general knowledge in the relevant field or is otherwise admissible prior art in relation to the validity of the claims.

1. A system for remote groundwater monitoring, the system including
one or more sensor modules configured for distribution in one or more groundwater monitoring wells, each sensor module being adapted to acquire multi-parameter sensor data from each groundwater monitoring well, wherein the multi-parameter sensor data includes electrochemical property data and electrical property data of groundwater in each groundwater monitoring well,
one or more hubs coupled to the one or more sensor modules for retrieving the multi-parameter sensor data and wirelessly communicating with an online server to upload the multi-parameter sensor data to the online server.

2. A system of claim 1, wherein the one or more groundwater monitoring wells are located in or around an in-situ recovery mine site, and the one or more sensor modules are configured to be submerged underwater to acquire multi-parameter sensor data from an aquifer layer of the groundwater monitoring wells.

3. A system of claim 1, wherein each sensor module includes a plurality of solid-state sensors comprising
a pH electrode, and
a reference electrode,
for acquiring data relating to electrochemical properties of the groundwater in each groundwater monitoring well.

4. A system of claim 3, wherein each sensor module further includes an oxidation reduction potential electrode, for acquiring further data relating to electrochemical properties of the groundwater in each groundwater monitoring well.

5. A system of claim 3, wherein each sensor module further includes an electrical conductivity sensor for acquiring data relating to electrical properties of the groundwater in each groundwater monitoring well.

6. A system of claim 3, wherein each sensor module further includes a temperature sensor and/or a water pressure sensor.

7. A system of claim 1, wherein each sensor module is configured for connection with one or more self-contained sensor units, and wherein the one or more sensor units includes a water pressure sensor unit.

8. A system of claim 7, further including
a barometer located within 20 km of the one or more groundwater monitoring wells for measuring atmospheric pressure, the barometer being configured for communication with the online server, the system being configured to determine the water level in the one or more groundwater monitoring wells based on the measurements from the barometer and the one or more water pressure sensors,
or
a reference water pressure sensor for distribution in the one or more groundwater monitoring wells such that the reference water pressure sensor is suspended above groundwater, the reference water pressure sensor being coupled to the hub for wireless communication with the online server, the system being configured to determine the water level in the one or more groundwater monitoring wells based on the measurements from the reference water pressure sensor and the one or more water pressure sensors.

9. A system of claim 1, wherein the one or more hubs are adapted to periodically sample sensor data from each sensor module for wireless communication to the online server in real-time or near real-time.

10. A system of claim 1, wherein each sensor module is configured to measure electrical conductivity of groundwater and electrochemical properties of groundwater in each respective groundwater monitoring well at non-overlapping time intervals.

11. A system of claim 1, wherein each sensor module is associated with a spacer for spacing a sensor face of the sensor module at a predetermined distance away from a wall of a respective groundwater well.

12. A system of claim 1, wherein the hub is configured to
provide power to the one or more sensor modules,
schedule sampling of the one or more sensor modules,
buffer sensor data from the one or more sensor modules, and
upload the sensor data to the online server, and
wherein the online server is configured to
receive sensor data from the hub,
store the received sensor data in a database, and
provides online access of the sensor data to a remote user.

13. A groundwater monitoring system including
one or more sensor modules configured for distribution in one or more groundwater monitoring wells, the groundwater monitoring wells being located in or around an in-situ recovery mine site,
the one or more sensor modules being configured to be submerged underwater to measure multi-parameter sensor data from an aquifer layer of the groundwater monitoring wells, wherein the multi-parameter sensor data includes electrochemical property data and electrical property data of groundwater in each groundwater monitoring well,
one or more hubs coupled to the one or more sensor modules, the hubs being configured to periodically sample the multi-parameter sensor data measured by the one or more sensor modules, and
an online server for receiving and storing the sensor data uploaded from the hub and providing online access to the sensor data.

14. The system of claim 13, wherein each sensor module is configured to measure electrical conductivity of groundwater and electrochemical properties of groundwater in each respective groundwater monitoring well at non-overlapping time intervals.

15. The system of claim 13, wherein each sensor module includes one or more sensors for measuring electrochemical properties of groundwater in each groundwater monitoring well, and an electrical conductivity sensor for measuring conductivity of the groundwater in each groundwater monitoring well, wherein the sensors for measuring electrochemical properties and the electrical conductivity sensor is embedded in the sensor module.

16. The system of claim 15, wherein the one or more sensors for measuring electrochemical properties include
a pH electrode, and
a reference electrode,
for acquiring data relating to electrochemical properties of the groundwater in each groundwater monitoring well.

17. The system of claim 16, wherein each sensor module further includes an oxidation reduction potential electrode.

18. A sensor module for groundwater monitoring, the sensor module being configured to be submerged underwater to measure multi-parameter sensor data from an aquifer layer of groundwater monitoring wells in or around an in-situ recovery mine site, wherein the sensor module includes one or more sensors for measuring electrochemical properties of groundwater in each groundwater monitoring well, and an electrical conductivity sensor for measuring electrical conductivity of the groundwater in each groundwater monitoring well, wherein the sensors for measuring electrochemical properties and the electrical conductivity sensor is embedded in the sensor module.

19. The sensor module of claim 18, wherein the sensor module is configured to measure electrical conductivity of groundwater and electrochemical properties of groundwater in each respective groundwater monitoring well at non-overlapping time intervals.

20. The sensor module of claim 18, wherein the one or more sensors for measuring electrochemical properties includes
a pH electrode, and
a reference electrode,
for acquiring data relating to electrochemical properties of the groundwater in each groundwater monitoring well.
